(19) United States
(12) Patent Application Publication
(10) Pub. No.: US 2026/0185149 A1
(43) Pub. Date: Jul. 2, 2026

(54) DETECTION METHOD, COMPUTER PROGRAM PRODUCT, DATA PROCESSING UNIT AND DETECTION SYSTEM FOR DETECTING MUTATIONS OF A POLYNUCLEOTIDE IN A BIOLOGICAL SAMPLE

(71) Applicant: ONCOBIT AG, Schlieren (CH)

(72) Inventor: Lucas Kohler, Zurich (CH)

(21) Appl. No.: 19/111,965

(22) PCT Filed: Sep. 20, 2023
(86) PCT No.: PCT/EP2023/075964

§ 371 Date: Mar. 14, 2025

(30) Foreign Application Priority Data
Sep. 28, 2022 (CH) CH001119/2022

(51) Int. Cl. C12Q 1/6851

(52) U.S. Cl. CPC C12Q 1/6851

(57) ABSTRACT

One aspect of the disclosure relates to a detection method for reliably detecting mutations of at least one target polynucleotide in a biological sample. The method comprises the step of providing in the same batch and from a common sample plate a control sample and the biological sample to a dPCR unit. The detection method further comprises the steps of generating a biological fluorescence data set using the biological sample and a control fluorescence data set using the control sample and obtaining by a fluorescence data processing unit the biological fluorescence data set and the control fluorescence data set generated by the dPCR unit. The detection method comprises the step of detecting in the fluorescence data processing unit from the biological fluorescence data set mutant copies in the biological sample using the control fluorescence data set.

### FIELD OF THE DISCLOSURE

The present disclosure relates to a detection method, a computer program product, a fluorescence data processing unit and a detection system for detecting mutations of at least one polynucleotide in a biological sample.

### BACKGROUND OF THE DISCLOSURE

WO2016197028A1 published in December 2016 in the name of Life Technologies Corp., relates to a method for determining false positives calls in a biological data plot. The method includes identifying a first data cluster as non-amplification data points within the biological data plot and identifying a second data cluster as wild-type positives within the biological data plot. The method further includes estimating a position in the biological data plot of a center of the first and second data clusters. The method further includes determining, for each data point within the first data cluster, a probability of belonging to the first data cluster and determining, for each data point within the second data cluster, a probability of belonging to the second data cluster. The method includes applying a probability threshold for each data point within the first and second data cluster to identify false positives.

CN111235240A published in June 2020 in the name of Guangzhou Forevergen Biotechnology Co Ltd and Guangdong Yongnuo Medical Tech Co Ltd, relates to PCR reaction liquid and kit for detecting mutation of a V600E site of a human BRAF gene. The kit comprises the PCR reaction liquid. The PCR reaction liquid comprises a PCR reaction pre-mixed solution, an upstream primer Pri-F1, a downstream Pri-R1, a wild probe and a mutation probe. The final concentrations and specificities of the adopted primers and probes are particularly suitable for digital PCR detection, and a digital PCR technique is utilized for detection.

### SUMMARY OF THE DISCLOSURE

Liquid biopsies provide the ability to monitor solid tumors in patients, allowing, e.g. to assess the success of an ongoing cancer therapy. However, the levels of cell free DNA (cfDNA) in liquid biopsies can be low, and the levels of circulating tumor DNA (ctDNA) with a mutation are typically even lower. Detecting tumor-specific mutations in ctDNA is difficult and only possible with very sensitive and specific detection methods. Digital polymerase chain reaction (dPCR) is a quantitative PCR (qPCR) technology that allows for the detection, identification and quantification of specific DNA markers in biological samples, in particular in highly diluted biological samples such as plasma. The following disclosure is made with reference to droplet digital polymerase chain reaction (ddPCR) primarily, however other digital PCR methods and devices/units are equally suitable, e.g. solid compartment-based PCR methods and devices can be used. In dPCR approaches, a sample is generally split into thousands of partitions, which serve as individual reaction compartments. These partitions can be liquid compartments (e.g., droplets in ddPCR) or solid compartments (e.g., wells and microchambers in nanoplate, chip-based or microfluidic chamber-based dPCR).

For analysis, the same reagents/assays as for qPCR are used, but the sample (and therewith the polynucleotide molecules (DNA)) is split into partitions (e.g., droplets) prior to amplification, with the objective of getting up to one target polynucleotide per partition. The fluorescence signal from all partitions (e.g., droplets) are read out after the amplification process. dPCR devices such as ddPCR devices, often use dual-channel fluorescent probes to quantify target polynucleotide (DNA) molecules in said biological samples. The two channels are typically used to measure a wild-type and at least one mutant variant of the target polynucleotide. Based on the read-out fluorescence signals it should be determined if and in how many partitions (e.g. droplets) the target the wild-type polynucleotide and in how many the mutant polynucleotide was amplified. Preferably, a concentration of mutant copies of the target polynucleotide is determined. The concentration of mutant copies of the target polynucleotide typically refers to the ratio of mutant copies to total copies (mutant and wild-type copies) of the target polynucleotide in a sample. Based on this, a biological sample can be classified as including mutations of at least one target polynucleotide. However, known methods do not take inter-batch and inter-plate variations of the dPCR into account, which can affect the fluorescence signals in the wild-type and in the mutant channel respectively. Detection methods should thus provide detection results in a reliable and robust manner.

It is an object of this disclosure to provide a detection method, a computer program product, a fluorescence data processing unit and a detection system for detecting mutations of at least one polynucleotide in a biological sample. In particular, it is an object of the present disclosure to provide a detection method, a computer program product, a fluorescence data processing unit and a detection system for reliably detecting mutations of at least one polynucleotide in a biological sample, which detection method addresses at least one of the disadvantages of the prior art. According to the present disclosure, these objects are achieved by the features of the independent claims. In addition, further advantageous embodiments follow from the dependent claims and the description.

In a preferred variation, a first aspect of the disclosure is directed to a detection method for reliably detecting mutations of at least one target polynucleotide in a biological sample, in particular for reliably quantifying low concentration mutations of at least one target polynucleotide in the biological sample. The method comprises the step of providing in the same batch and/or from a common sample plate a control sample and the biological sample to a digital polymerase chain reaction (dPCR) unit. In a more preferred variation, a droplet digital polymerase chain reaction (ddPCR) unit is utilized. Alternatively, a solid compartment-based digital PCR unit can be used. The control sample usually comprises a predefined amount of mutant and wild-type copies of the target polynucleotide. When providing the control sample and the biological sample in the same batch and/or from a common sample plate, the environmental factors for both samples are essentially identical during the PCR process. The detection method comprises the steps of generating by the dPCR unit a biological fluorescence data set using the biological sample and a control fluorescence data set using the control sample and obtaining by a fluorescence data processing unit the biological fluorescence data set and the control fluorescence data set generated by the dPCR unit. Usually, each data set comprises a multiplicity of fluorescence points respectively having an intensity value in a wild-type channel and in a mutant channel of the target polynucleotide. Each fluorescence point typically corresponds to the fluorescence signal of a partition (e.g., droplet) read out by the dPCR unit. A sample plate typically refers to a multi-well plate for accommodating a sample in a well (multiple samples that are analyzed together are analyzed on a common sample plate; inter-plate variation generally refers to the variation related to the analyses of samples on separate sample plates). In some variations semi-skirted PCR plates can be used as sample plates.

The detection method typically comprises detecting in the fluorescence data processing unit from the biological fluorescence data set mutant copies in the biological sample using the control fluorescence data set. Using the control fluorescence data set, obtained in the dPCR unit from the control sample comprising predefined amounts of mutant and wild-type copies under the essentially same conditions as the biological fluorescence data set, to detect mutant copies in the biological fluorescence data set, allows to mitigate inter-batch and/or inter-plate variations. This increases the reliability and the robustness of the detection method.

Depending on the field of application, the detection method comprises, alternatively or in addition, the step of determining in the fluorescence data processing unit from the biological fluorescence data set an amount of mutant and wild-type copies respectively and/or a concentration of mutant copies of the target polynucleotide using the control fluorescence data set.

Preferably the detection method comprises the steps of determining by one or more processors of the fluorescence data processing unit a mutant threshold value using the control fluorescence data set. Alternatively or in addition, the one or more processors of the fluorescence data processing unit determine a wild-type threshold value using the control fluorescence data set. These thresholds are typically used for determining, if a fluorescence point is positive in the respective channel, meaning that in the corresponding partition (e.g., droplet) of the dPCR unit was at least one copy of the polynucleotide (mutant and/or wild-type). A fluorescence point is usually considered positive, when its intensity value is the respective channel is above the respective threshold.

In some variations, the one or more processors of the fluorescence data processing unit determine the number of fluorescence points of the biological fluorescence data set having an intensity value in the mutant channel above the mutant threshold. In addition, they may determine the number of fluorescence points of the biological fluorescence data set having an intensity value in the wild-type channel above the wild-type threshold.

To account for multiple polynucleotide molecules (mutant and/or wild-type) per partition (e.g. droplet), a Poisson correction should be calculated. In some variations, the one or more processors of the fluorescence data processing unit calculate a Poisson correction of the number of fluorescence points of the biological fluorescence data set having an intensity value in the mutant channel above the mutant threshold. In addition, they may calculate a Poisson correction of the number of fluorescence points of the biological fluorescence data set having an intensity value in the wild-type channel above the wild-type threshold. This way an amount/number of mutant copies and/or wild-type copies can be determined from the number of fluorescence points being positive in the mutant and/or in the wild-type channel. The mutant concentration (ratio of mutant copies to wild-type copies) can be outputted by the fluorescence data processing unit.

Depending on the field of application, the one or more processors of the fluorescence data processing unit compare the determined number and/or concentration of mutant copies to a predefined limit of blank (lob) value for classifying the biological sample. In case the concentration exceeds the lob value, the biological sample is usually classified as positive for mutations of the target polynucleotide. The lob value can be understood as the lower bound of the number and/or concentration of mutant copies of the target polynucleotide for classifying the biological sample as including mutations of the target polynucleotide.

Alternatively, or in addition, the one or more processors of the fluorescence data processing unit determine using the control fluorescence data set an expected range for at least one of the following: the clusters and the cluster centers of mutant positive and wild-type negative partitions (e.g. droplets), wild-type positive and mutant negative partitions (e.g. droplets), and mutant negative and wild-type negative partitions (e.g. droplets), respectively. The expected ranges preferably comprise a least one boundary in the mutant and/or the wild-type channel, in particular an upper and a lower boundary.

For a reliable performance, the detection method comprises the steps of determining by one or more processors of the fluorescence data processing unit a cross-reactivity threshold value using the control fluorescence data set. Cross-reactivity is typically indicated by partitions (e.g., droplets) (fluorescence points) or a cluster center of partitions (e.g., droplets) lying below the expected range and is detected by counting the number of partitions (e.g. droplets) in different ranges of signal intensities. If the number of (mutant/wild-type) positive partitions (e.g., droplets) below the (mutant/wild-type) cross-reactivity threshold value is higher than the number of (mutant/wild-type) partitions (e.g. droplets) above, the sample is likely to be cross-reactive. Alternatively, or in addition, if a cluster of partitions (e.g., droplets) above the mutant negative and wild-type negative cluster and below the (mutant/wild-type) positive cluster and/or the expected range of the (mutant/wild-type) cluster center is detected, the sample is likely to be cross-reactive.

In a preferred variation, the one or more processors of the fluorescence data processing unit determine the number of fluorescence points of the biological fluorescence data set having an intensity value in the mutant channel above the mutant threshold and below the cross-reactivity threshold value, so called weakly positive partitions (e.g., droplets). In particular, the number of weakly positive partitions (e.g., droplets) (fluorescence points between the mutant threshold and the cross-reactivity threshold value) is compared to the number strongly positive partitions (e.g. droplets) (fluorescence points above the cross-reactivity threshold value). Preferably the one or more processors of the fluorescence data processing unit issue a warning in case the number of weakly positive partitions (e.g. droplets) is greater than the number of strongly positive partitions (e.g. droplets). The same cross-reactivity checks can analogously be performed in the wild-type channel. A combination of cross-reactivity checks is possible as well.

In a preferred variation, the one or more processors of the fluorescence data processing unit determine a density of partitions (e.g., droplets) of wild-type negative (and mutant-negative) partitions (e.g. droplets). In particular, this density is examined for having unexpected clusters above the mutant negative/wild-type negative cluster and below the mutant (wild-type) positive cluster by determining peaks in the density between said clusters. Alternatively, or in addition, unexpected clusters can be found in the expected range of the (mutant/wild-type) cluster center by determining peaks in the density between said clusters. Usually more than two clusters in one channel are not expected, thus more than two clusters are indicative for a cross-reactive sample or other issues. Preferably the one or more processors of the fluorescence data processing unit issue a warning in case such a cluster is found.

In some variations, the detection method comprises the steps of identifying by the one or more processors of the fluorescence data processing unit from the control fluorescence data set in the mutant channel a mutant positive cluster of fluorescence points and mutant negative cluster of fluorescence points and deriving the mutant threshold based on said clusters. Alternatively, or in addition, the one or more processors of the fluorescence data processing unit identify from the control fluorescence data set in the wild-type channel a wild-type positive cluster of fluorescence points and wild-type negative cluster of fluorescence points and deriving the wild-type threshold based on said clusters.

Good results are possible when the one or more processors of the fluorescence data processing unit determine the mutant threshold value and/or the cross-reactivity threshold value based on at least one of the following. A center of the mutant positive cluster and a center of the mutant negative cluster respectively and a distribution of the fluorescence point in the mutant positive cluster and/or a distribution of the fluorescence point in the mutant negative cluster, wherein the distributions are in particular a fitted distribution. If appropriate, a continuous probability distribution is used to fit the partitions (e.g., droplets) (fluorescence data points), such as a log-normal distribution.

Analogously the detection method may comprise determining by the one or more processors of the fluorescence data processing unit the wild-type threshold value and/or the cross-reactivity wild-type threshold value based on at least one of the following. A center of the wild-type positive cluster and/or a center of the wild-type negative cluster respectively and a distribution of the fluorescence point in the wild-type positive cluster and/or a distribution of the fluorescence point in the wild-type negative cluster, wherein the distributions are in particular a fitted distribution.

Depending on the configuration, an initial mutant threshold is calculated as a certain quantile of the fitted continuous probability distribution or of the discrete distribution of the partitions (e.g., droplets). The control sample is designed to have a large amount of partitions (e.g. droplets) (fluorescence points) comprising wild-type polynucleotides. The distribution is fitted to this cluster and the quantile is predefined accordingly large, in particular between 1-1e-4 and 1-1e-6, preferably around 1-1e-5. Said quantile can be used as an initial mutant threshold. Preferably, the mutant threshold value is calculated based on the initial mutant threshold and the center of the mutant positive cluster. In particular the mutant threshold is calculated as a weighted mean between the initial mutant threshold and the center of the mutant positive cluster, preferably with weights 2:1. In an analogous manner the (mutant) cross-reactivity threshold can be calculated as weighted mean between the initial mutant threshold and the center of the mutant positive cluster, preferably with weights 1:3. Depending on the field of application the weights may be selected differently without departing from the disclosure.

If appropriate, the detection method comprises the steps of determining by the one or more processors of the fluorescence data processing unit a result for the biological sample. In particular the result is determined by comparing the amount and/or concentration of mutant copies to a pre-defined lob value. In case the amount and/or the concentration of mutant copies is above the pre-defined lob value, a “positive” result value can be assigned to the pathological sample. The result comprises typically at least one of the following, the amount of mutant copies, the concentration of mutant copies, the result value, a confidence value or interval, and a recommendation of at least one specific test. The recommendation of at least one specific test preferably depends on the result value. In a case a “positive” result value is determined, a specific test for validating the result value can form part of the result. The specific test recommendation may depend on the field of application, in some cases it can be a Computer Tomography (CT) scan of the origin of the biological sample, in particular a Positron-Emissions-Tomography (PET) CT scan thereof.

Preferably the method further includes displaying by a display interconnected to the fluorescence data processing unit the result. Alternatively, or in addition, the method includes at least one of the following, storing the result on a storage medium, printing the result at printer interconnected to the fluorescence data processing unit and transmitting the result to a communication device interconnected to the fluorescence data processing unit via a communication network.

A second aspect of the disclosure is directed to a computer program product comprising a non-transitory computer readable medium having stored thereon computer program code configured to direct one or more processors, in particular of a fluorescence data processing unit for obtaining by a fluorescence data processing unit the biological fluorescence data set and the control fluorescence data set generated by the dPCR unit, each data set comprising a multiplicity of fluorescence points respectively having an intensity value in a wild-type channel and in a mutant channel of the target polynucleotide; and detecting in the fluorescence data processing unit from the biological fluorescence data set mutant copies in the biological sample using the control fluorescence data set.

In a preferred variation the computer readable medium has stored thereon further computer program code configured to direct one or more processors of a fluorescence data processing unit for determining in the fluorescence data processing unit from the biological fluorescence data set an amount of mutant and wild-type copies respectively and/or a concentration of mutant copies of the target polynucleotide using the control fluorescence data set.

Depending on the design, the computer readable medium has stored thereon further computer program code configured to direct one or more processors of a fluorescence data processing unit for determining a mutant threshold value and/or a wild-type threshold value from the fluorescence data obtained from the control sample. Further, the one or more processors can be directed by the computer program code for determining the number of fluorescence points obtained from the biological sample having an intensity value in the mutant channel above the mutant threshold and determining the number of fluorescence points obtained from the biological sample having an intensity value in the wild-type channel above the wild-type threshold.

For an increased reliability, the computer readable medium has stored thereon further computer program code configured to direct one or more processors of a fluorescence data processing unit for determining a cross-reactivity threshold value using the control fluorescence data set. And in turn for determining a number of weakly positive fluorescence points as the number of fluorescence points of the biological fluorescence data set having an intensity value in the mutant channel above the mutant threshold and below the cross-reactivity threshold value, and determining the number strongly positive fluorescence points as the number of fluorescence points of the biological fluorescence data set having an intensity value in the mutant channel above the cross-reactivity threshold. Preferably in addition, comparing the number of weakly positive fluorescence points to the number strongly positive fluorescence points to evaluate if the biological sample is cross-reactive.

Preferably the computer readable medium has stored thereon further computer program code configured to direct one or more processors of a fluorescence data processing unit for identifying from the fluorescence data obtained from the control sample in the mutant channel a mutant positive cluster of fluorescence points and mutant negative cluster of fluorescence points and deriving the mutant threshold based on said clusters. Alternatively, or in addition, the one or more processors can be directed by the computer program code for identifying from the fluorescence data obtained from the control sample in the wild-type channel a wild-type positive cluster of fluorescence points and wild-type negative cluster of fluorescence points and deriving the wild-type threshold based on said clusters.

Good results are possible when the computer readable medium has stored thereon further computer program code configured to direct one or more processors of a fluorescence data processing unit for determining by the one or more processors of the fluorescence data processing unit the mutant threshold based on at least one of the following. A center of the mutant positive cluster and a center of the mutant negative cluster respectively and a distribution of the fluorescence point in the mutant positive cluster and/or a distribution of the fluorescence point in the mutant negative cluster, wherein the distributions are in particular a fitted distribution.

Analogously the computer readable medium has stored thereon further computer program code configured to direct one or more processors of a fluorescence data processing unit for determining by the one or more processors of the fluorescence data processing unit the wild-type threshold based on at least one of the following. A center of the wild-type positive cluster and/or a center of the wild-type negative cluster respectively and a distribution of the fluorescence point in the wild-type positive cluster and/or a distribution of the fluorescence point in the wild-type negative cluster, wherein the distributions are in particular a fitted distribution.

The computer readable medium has preferably stored thereon further computer program code configured to direct one or more processors of a fluorescence data processing unit for determining a result for the biological sample, in particular for determining a result value by comparing the amount and/or the concentration of mutant copies to a predefined lob value, said result comprising at least one of the following, the amount of mutant copies, the concentration of mutant copies, a result value, a confidence value or interval, and a recommendation of at least one specific test; and displaying on a display interconnected to the fluorescence data processing unit the result. The lob value typically defines the lower bound of the amount and/or concentration of mutant copies of the target polynucleotide for classifying the biological sample as including mutations of the target polynucleotide.

Another aspect of the disclosure is directed to a fluorescence data processing unit for use in a detection method as described above. The fluorescence data processing unit typically comprises one or more processors configured for obtaining by a biological fluorescence data set and a control fluorescence data set generated by a dPCR unit, each data set comprising a multiplicity of fluorescence points respectively having an intensity value in a wild-type channel and in a mutant channel of the target polynucleotide. In addition, the one or more processors are preferably configured for detecting from the biological fluorescence data set mutant copies in the biological sample using the control fluorescence data set and/or determining from the biological fluorescence data set an amount of mutant and wild-type copies and/or a concentration of mutant copies of the target polynucleotide using the control fluorescence data set.

Good performance is possible when the fluorescence data processing unit comprises a memory having stored thereon a lob value for assigning a result value to the biological sample, said lob value defining lower bound of the amount and/or concentration of mutant copies of the target polynucleotide for classifying the biological sample as including mutations of the target polynucleotide.

Another aspect of the disclosure is directed to a detection system comprising a dPCR unit and a fluorescence data processing unit for carrying out the detection method as described above. The previously described embodiments of the method for reliably detecting mutations of at least one target polynucleotide in a biological sample disclose at the same time correspondingly designed embodiments of the device and vice versa.

While the terms “droplet digital PCR” and “ddPCR” are trademarks of Bio-Rad Laboratories Inc. and the technologies it refers to falls within the meaning of ddPCR of the present disclosure, terms are not limited thereto.

### BRIEF DESCRIPTION OF THE DRAWINGS

The present disclosure will be explained in more detail, by way of example, with reference to the drawings in which:

FIG. 1 shows a block diagram illustrating schematically a detection method and a variation of a fluorescence data processing unit according to the disclosure.

FIG. 2 shows a flow diagram illustrating an exemplary sequence of steps for a detection method according to the disclosure.

FIG. 3 shows a flow diagram illustrating an exemplary sequence of steps for a detection method according to the disclosure.

FIG. 4 shows a scatter plot of fluorescence points of an exemplary biological fluorescence data set.

### DETAILED DESCRIPTION OF THE EMBODIMENTS

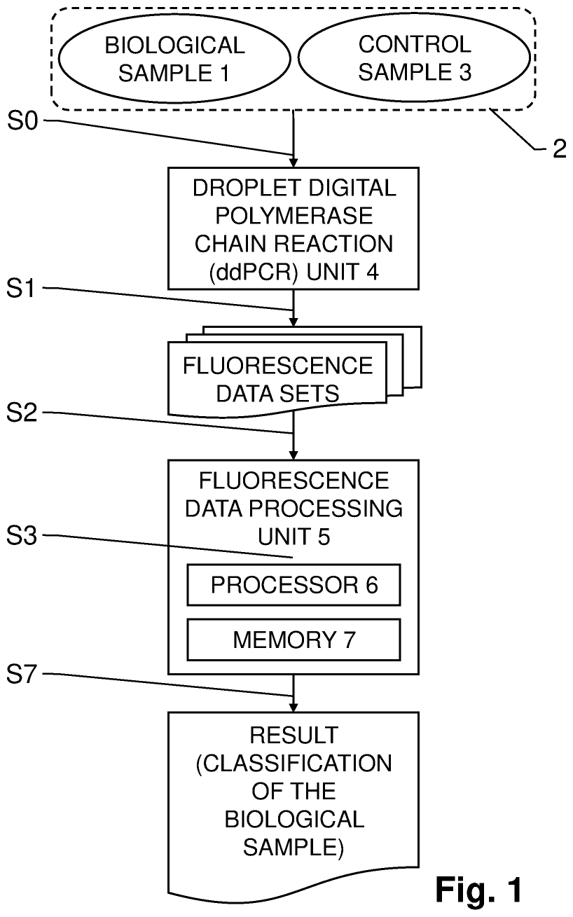

In the following paragraphs, described with reference to FIGS. 1 to 3 are possible sequences of steps of the variation of detection method. FIG. 1 illustrates an outline of a detection method for reliably detecting mutations of at least one target polynucleotide in a biological sample 1 according to the disclosure. In preparatory step S0, a control sample 3 and a biological sample 1 are provided to a droplet digital polymerase chain reaction (ddPCR) unit 4.

In step S1, the ddPCR unit 4 generates a biological fluorescence data set using the biological sample 1. The ddPCR unit 4 further generates a control fluorescence data set using the control sample 3. The samples are respectively and therewith the target polynucleotide molecules split into many thousands of droplets, with the objective of getting just one target polynucleotide per droplet. Fluorescence signals in a mutant channel and in a wild-type channel from all droplets are read out after the amplification process for forming the biological fluorescence data set and a control fluorescence data set.

In step S2, a fluorescence data processing unit 5 obtains the biological fluorescence data set and the control fluorescence data set generated by the ddPCR unit 4, each data set comprising a multiplicity of fluorescence points respectively having an intensity value in a wild-type channel and in a mutant channel of the target polynucleotide. The fluorescence data processing unit 5 may read the data sets from a storage device interconnected to the fluorescence data processing unit 5. In other variations the fluorescence data processing unit 5 is interconnected locally or remotely via a communication network to the ddPCR unit 4 to receive the data sets therefrom. The fluorescence data processing unit 5 can be implemented as a mobile processing unit, such as a laptop, a tablet etc. In other variations the fluorescence data processing unit 5 is implemented as a remote processing unit such as a cloud computing unit.

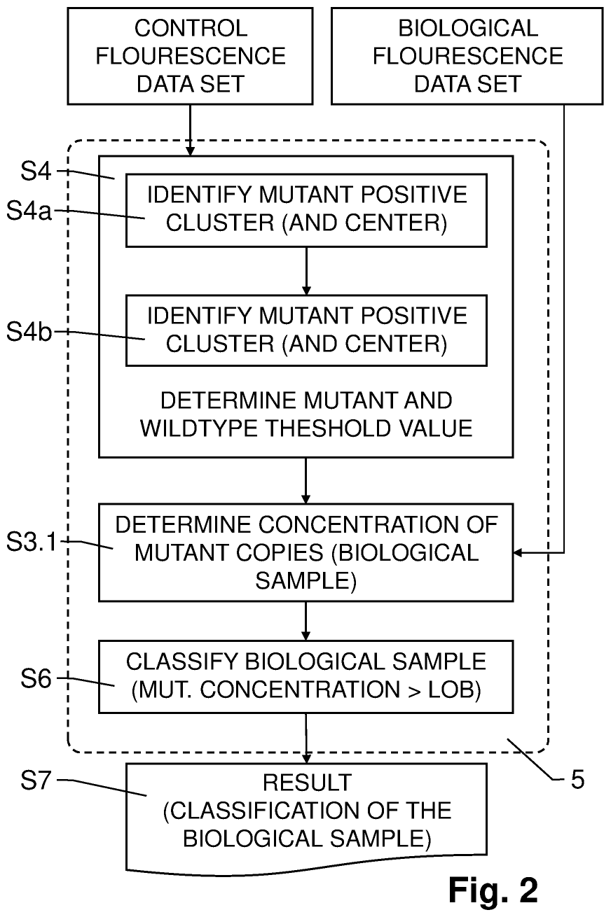

In step S3, the fluorescence data processing unit 5 detects from the biological fluorescence data set mutant copies in the biological sample 1 using the control fluorescence data set. As shown in FIG. 2, the fluorescence data processing unit 5 may determine in step S3.1 alternatively or in addition, from the biological fluorescence data set an amount of mutant and wild-type copies respectively and/or a concentration of mutant copies of the target polynucleotide using the control fluorescence data set. In order to achieve this, the processor 6 of the fluorescence data processing unit 5 determines the number of fluorescence points 8 of the biological fluorescence data set having an intensity value in the mutant channel above the mutant threshold 9, and determining the number of fluorescence points 8 of the biological fluorescence data set having an intensity value in the wild-type channel above the wild-type threshold 10.

In step S6, the processor 6 of the fluorescence data processing unit 5 assigns a result value to the biological sample 1 by comparing the amount and/or the concentration of mutant copies to a predefined lob value stored in a memory 7 of the fluorescence data processing unit 5. In step S7 the result is outputted, this can for example be achieved by displaying the result on a display (not shown) interconnected to the fluorescence data processing unit 5 or incorporated into the fluorescence data processing unit 5.

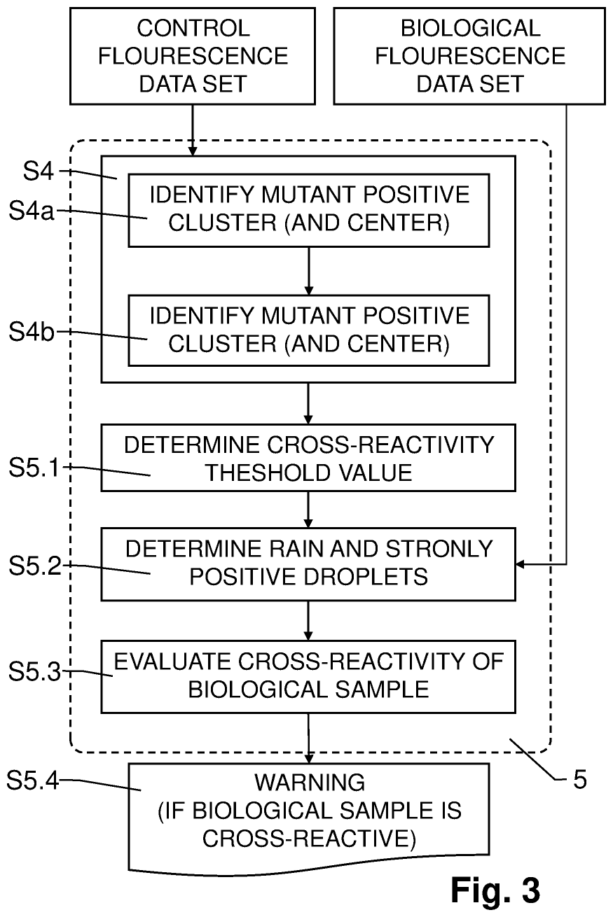

As visible in FIG. 3, the shown variation of the disclosure comprises in step S5.1 determining by processor 6 of the fluorescence data processing unit 5 a cross-reactivity threshold 11 value using the control fluorescence data set. Based thereon, the processor determines in step S5.2 a number of weakly positive fluorescence points 8 as the number of fluorescence points 8 of the biological fluorescence data set having an intensity value in the mutant channel above the mutant threshold 9 and below the cross-reactivity threshold 11 value, and a number of strongly positive fluorescence points 8 as the number of fluorescence points 8 of the biological fluorescence data set having an intensity value in the mutant channel above the cross-reactivity threshold 11. The cross-reactivity threshold can be calculated for the mutant channel 11 and/or for the wild-type channel 16. By comparing the number of weakly positive fluorescence points 8 to the number of strongly positive fluorescence points 8 in step S5.3 the fluorescence data processing unit 5 is able to evaluate if the biological sample is cross-reactive. In particular, if there are more weakly positive fluorescence points 8 than strongly positive fluorescence points 8 the biological sample is considered cross-reactive. Alternatively, or in addition, if a cluster of droplets accumulating above the mutant negative and wild-type negative cluster 13 and below the mutant or wild-type positive cluster 14 and/or the expected range of the (mutant/wild-type) cluster center 15 is detected, the sample 3 is likely to be cross-reactive. To determine the presence of unexpected clusters the fluorescence data processing unit 5 determine a density of droplets 8 of wild-type negative (mutant-negative) droplets can be determined. In particular, this density may be examined for having unexpected clusters above the mutant/wild-type negative cluster 13 and below the mutant (wild-type) positive cluster 12 (14) and/or the expected range of the (mutant/wild-type) cluster center 15 by finding peaks in the density.

In step S5.4 a warning is issued by the fluorescence data processing unit 5. The warning is typically outputted the same way as the results or together therewith.

In a preferred variation of the disclosure, the steps for determining the thresholds are as follows.

For each Assay A and Batch B on the sample plate:
For each Positive Control sample (control fluorescence data set):
   Identify clusters and cluster centers
      K-means algorithm to split through mutant channel in 1-dimension (S4a)
      K-means algorithm to split through wild-type channel in 1-dimension (S4b)
      Calculate cluster centers 15 as medians of the respectively assigned droplets
   Identify thresholds for mutant channel (S4, S4a)
      Fit the wild-type positive cluster 14 with a 1-dimensional log-normal distribution along mutant channel.
      Compute the initial mutant threshold as the (1-1e-5) quantile of the log-normal distribution
      Compute the mutant threshold as a weighted mean (2:1) between the initial mutant threshold and the mutant positive cluster 12 center
      Compute the cross-reactivity threshold 11 as a weighted mean (1:3) between the initial mutant threshold and the mutant positive cluster 12 center
   Identify thresholds for wild-type channel (S4, S4b).
      Fit the wild-type negative cluster 13 with a 1-dimensional log-normal distribution along wild-type channel.
      Compute the initial wild-type threshold as the (1-1e-5) quantile of the log-normal distribution
      Compute the wild-type threshold as a weighted mean (2:1) between the initial wild-type threshold and the wild-type positive cluster 14 center
      Compute the cross-reactivity threshold 16 as a weighted mean (1:3) between the initial wild-type threshold and the wild-type positive cluster 14 center
Derive the final channel thresholds by taking the median over all Positive Control samples' thresholds.

In a preferred variation of the disclosure, the steps for classifying a biological sample based on the determined thresholds are as follows.

For each biological sample (biological fluorescence data set):
For each droplet in the biological fluorescence data set:
   Classify the droplet into a cluster (NN—mutant and wild-type negative 13, PN—mutant positive 12, NP—wild-type positive 14, PP—mutant and wild-type positive) by applying the respective thresholds (S6)
For each channel (S3.1):
   Compute the number of mutant and wild-type positive polynucleotides as the Poisson corrected numbers of positive droplets
   Compute the mutant concentration as 100*(number of mutant positive polynucleotides)/(total number of polynucleotides), where the total number of polynucleotides is defined as the sum of the number of mutant positive polynucleotides and the number of wild-type positive polynucleotides.
Classify the biological sample (S6):
   If (amount and/or concentration of mutant copies >lob): positive
   Else: negative

It should be noted that, in the description, the sequence of the steps has been presented in a specific order, one skilled in the art will understand, however, that the order of at least some of the steps could be altered, without deviating from the scope of the disclosure. The same holds true for the computer program code that has been associated with specific functional modules in the description, that one skilled in the art will understand, the computer program code may be structured differently without deviating from the scope of the disclosure.

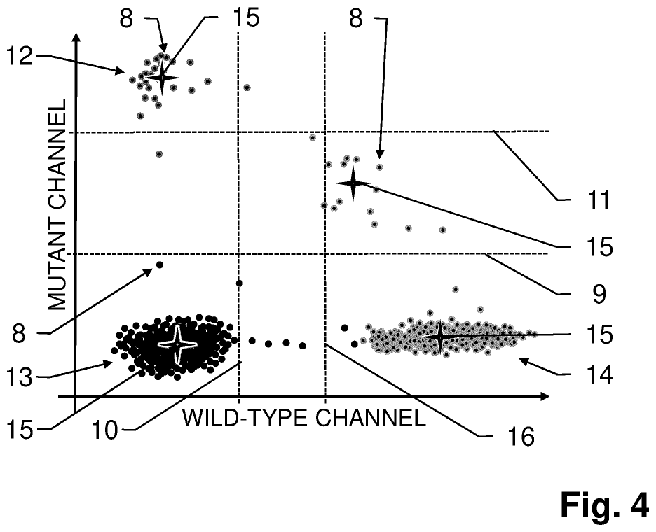

In FIG. 4 a typical scatter plot of a biological fluorescence data set is shown, with the following fluorescence point clusters: mutant/wild-type negative 13, mutant positive 12, wild-type positive 14 and mutant/wild-type positive. The respective clusters centers 15 are indicated by a cross symbol. Each threshold is indicated by a dashed line, the mutant threshold 9, the cross-reactivity threshold 11 (for the mutant channel), the cross-reactivity threshold 16 (for the wild-type channel) and the wild-type threshold 10.

1. A detection method for reliably detecting mutations of at least one target polynucleotide in a biological sample (1), the detection method comprising the steps of:
a. providing (S0) in the same batch and from a common sample plate (2) a control sample (3) and the biological sample (1) to a digital polymerase chain reaction (dPCR) unit (4), said control sample comprising a predefined amount of mutant and wild-type copies of the target polynucleotide;
b. generating (S1) by the dPCR unit (4) a biological fluorescence data set using the biological sample (1) and a control fluorescence data set using the control sample (3);
c. obtaining (S2) by a fluorescence data processing unit (5) the biological fluorescence data set and the control fluorescence data set generated by the dPCR unit (4), each data set comprising a multiplicity of fluorescence points (8) respectively having an intensity value in a wild-type channel and in a mutant channel of the target polynucleotide; and
d. detecting (S3) in the fluorescence data processing unit (5) from the biological fluorescence data set mutant copies in the biological sample (1) using the control fluorescence data set.

2. The detection method according to claim 1, wherein the detection method comprises the step of determining (S3.1) in the fluorescence data processing unit (5) from the biological fluorescence data set an amount of mutant and wild-type copies respectively and/or a (of mutant copies of the target polynucleotide using the control fluorescence data set.

3. The detection method according to claim 1, wherein the detection method comprises the steps of:
a. determining (S4) by one or more processors (6) of the fluorescence data processing unit (5) a mutant threshold (9) value and/or a wild-type threshold (10) value using the control fluorescence data set;
b. determining (S3.1) by the one or more processors (6) of the fluorescence data processing unit (5) the number of fluorescence points (8) of the biological fluorescence data set having an intensity value in the mutant channel above the mutant threshold (9), and determining the number of fluorescence points (8) of the biological fluorescence data set having an intensity value in the wild-type channel above the wild-type threshold (10).

4. The detection method according to claim 3, wherein the detection method comprises the steps of:
a. determining (S5.1) by one or more processors (6) of the fluorescence data processing unit (5) a cross-reactivity threshold (11) value using the control fluorescence data set;
b. determining (S5.2) by the one or more processors (6) of the fluorescence data processing unit (5) a number of weakly positive fluorescence points (8) as the number of fluorescence points (8) of the biological fluorescence data set having an intensity value in the mutant channel above the mutant threshold (9) and below the cross-reactivity threshold (11) value, and determining a number of strongly positive fluorescence points (8) as the number of fluorescence points (8) of the biological fluorescence data set having an intensity value in the mutant channel above the cross-reactivity threshold (11); and
c. comparing (S5.3) by the one or more processors (6) of the fluorescence data processing unit (5) the number of weakly positive fluorescence points (8) to the number strongly positive fluorescence points (8) to evaluate if the biological sample is cross-reactive.

5. The detection method according to claim 3, wherein the detection method comprises the steps of:
a. identifying (S4a) by the one or more processors (6) of the fluorescence data processing unit (5) from the control fluorescence data set in the mutant channel a mutant positive cluster (12) of fluorescence points (8) and mutant negative cluster (13) of fluorescence points (8) and deriving the mutant threshold (9) based on said clusters; and/or
b. identifying (S4b) by the one or more processors (6) of the fluorescence data processing unit (5) from the control fluorescence data set in the wild-type channel a wild-type positive cluster (14) of fluorescence points (8) and wild-type negative cluster (13) of fluorescence points (8) and deriving the wild-type threshold (10) based on said clusters.

6. The detection method according to claim 5, wherein the detection method comprises the steps of:
a. determining (S4a) by the one or more processors (6) of the fluorescence data processing unit (5) the mutant threshold (9) value and/or the cross-reactivity mutant threshold (11) value based on at least one of the following:
i. a center (15) of the mutant positive cluster (12) and a center (15) of the mutant negative cluster (13) respectively; and
ii. a distribution of the fluorescence point in the mutant positive cluster (12) and/or a distribution of the fluorescence point in the mutant negative cluster (13), wherein the distributions are a fitted distribution; and/or
b. determining (S4b) by the one or more processors (6) of the fluorescence data processing unit (5) the wild-type threshold (10) value and/or the cross-reactivity wild-type threshold value based on at least one of the following:
i. a center (15) of the wild-type positive cluster (14) and/or a center (15) of the wild-type negative cluster (13) respectively; and
ii. a distribution of the fluorescence point in the wild-type positive cluster (14) and/or a distribution of the fluorescence point in the wild-type negative cluster (13), wherein the distributions are a fitted distribution.

7. The detection method according to claim 1, wherein the detection method comprises the steps of:
a. determining (S6) by the one or more processors (6) of the fluorescence data processing unit (5) a result for the biological sample (1), in particular for determining a result value by comparing the amount and/or the concentration of mutant copies to a predefined limit of blank (lob) value, said result comprising at least one of the following, the amount of mutant copies, the concentration of mutant copies, the result value, a confidence value or interval, and a recommendation of at least one specific test; and
b. displaying (S7) by a display interconnected to the fluorescence data processing unit (5) the result.

8. A computer program product comprising a non-transitory computer readable medium having stored thereon computer program code configured to direct one or more processors (6) of a fluorescence data processing unit (5) for:
a. obtaining (S2) by a fluorescence data processing unit (5) the biological fluorescence data set and the control fluorescence data set generated by the dPCR unit (4), each data set comprising a multiplicity of fluorescence points (8) respectively having an intensity value in a wild-type channel and in a mutant channel of the target polynucleotide; and
b. detecting (S3) in the fluorescence data processing unit (5) from the biological fluorescence data set mutant copies in the biological sample (1) using the control fluorescence data set.

9. The computer program product of claim 8, wherein the computer readable medium has stored thereon further computer program code configured to direct one or more processors (6) of a fluorescence data processing unit (5) for determining (S3.1) in the fluorescence data processing unit (5) from the biological fluorescence data set an amount of mutant and wild-type copies respectively and/or a concentration of mutant copies of the target polynucleotide using the control fluorescence data set.

10. The computer program product of claim 8, wherein the computer readable medium has stored thereon further computer program code configured to direct one or more processors (6) of a fluorescence data processing unit (5) for:
a. determining (S4) a mutant threshold (9) value and/or a wild-type threshold (10) value from the fluorescence data obtained from the control sample (3);
b. determining (S3.1) the number of fluorescence points (8) obtained from the biological sample having an intensity value in the mutant channel above the mutant threshold (9), and determining the number of fluorescence points (8) obtained from the biological sample (1) having an intensity value in the wild-type channel above the wild-type threshold (10).

11. The computer program product of claim 10, wherein the computer readable medium has stored thereon further computer program code configured to direct one or more processors (6) of a fluorescence data processing unit (5) for:
a. determining (S5.1) a cross-reactivity threshold (11) value using the control fluorescence data set;
b. determining (S5.2) a number of weakly positive fluorescence points (8) as the number of fluorescence points (8) of the biological fluorescence data set having an intensity value in the mutant channel above the mutant threshold (9) and below the cross-reactivity threshold (11) value, and determining and determining a number of strongly positive fluorescence points (8) as the number of fluorescence points (8) of the biological fluorescence data set having an intensity value in the mutant channel above the cross-reactivity threshold (11); and
c. comparing (S5.3) the number of weakly positive fluorescence points (8) to the number strongly positive fluorescence points (8) to evaluate if the biological sample is cross-reactive.

12. The computer program product of claim 10, wherein the computer readable medium has stored thereon further computer program code configured to direct one or more processors (6) of a fluorescence data processing unit (5) for:
a. identifying (S4a) from the fluorescence data obtained from the control sample (3) in the mutant channel a mutant positive cluster (12) of fluorescence points (8) and mutant negative cluster (13) of fluorescence points (8) and deriving the mutant threshold (9) based on said clusters; and/or
b. identifying (S4b) from the fluorescence data obtained from the control sample (3) in the wild-type channel a wild-type positive cluster (14) of fluorescence points (8) and wild-type negative cluster (13) of fluorescence points (8) and deriving the wild-type threshold (10) based on said clusters.

13. The computer program product of claim 12, wherein the computer readable medium has stored thereon further computer program code configured to direct one or more processors (6) of a fluorescence data processing unit (5) for:
a. determining (S4a) by the one or more processors (6) of the fluorescence data processing unit (5) the mutant threshold (9) and/or the cross-reactivity mutant threshold (11) value based on at least one of the following:
i. a center (15) of the mutant positive cluster (12) and/or a center (15) of the mutant negative cluster (13) respectively; and
ii. a distribution of the fluorescence point in the mutant positive cluster (12) and/or a distribution of the fluorescence point in the mutant negative cluster (13), wherein the distributions are a fitted distribution; and/or
b. determining (S4b) by the one or more processors (6) of the fluorescence data processing unit (5) the wild-type threshold (10) and/or the cross-reactivity wild-type threshold value based on at least one of the following:
i. a center (15) of the wild-type positive cluster (14) and/or a center (15) of the wild-type negative cluster (13) respectively; and
ii. a distribution of the fluorescence point in the wild-type positive cluster (14) and/or a distribution of the fluorescence point in the wild-type negative cluster (13), wherein the distributions are a fitted distribution.

14. The computer program product of claim 8, wherein the computer readable medium has stored thereon further computer program code configured to direct one or more processors (6) of a fluorescence data processing unit (5)
a. determining (S6) a result for the biological sample (1), in particular for determining a result value by comparing the amount and/or the concentration of mutant copies to a predefined limit of blank (lob) value, said result comprising at least one of the following, the amount of mutant copies, the concentration of mutant copies, the result value, a confidence value or interval, and a recommendation of at least one specific test; and
b. displaying (S7) on a display interconnected to the fluorescence data processing unit (5) the result.

15. A fluorescence data processing unit (5) for use in a detection method according to claim 1, comprising one or more processors (6) configured for:
a. obtaining (S2) by a biological fluorescence data set and a control fluorescence data set generated by a dPCR unit (4), each data set comprising a multiplicity of fluorescence points (8) respectively having an intensity value in a wild-type channel and in a mutant channel of the target polynucleotide; and
b. detecting (S3) from the biological fluorescence data set mutant copies in the biological sample (1) using the control fluorescence data set; and/or
c. determining (S3.1) from the biological fluorescence data set an amount of mutant and wild-type copies respectively and/or a concentration of mutant copies of the target polynucleotide using the control fluorescence data set.

16. The fluorescence data processing unit (5) according to claim 15, wherein a memory (7) of the fluorescence data processing unit (5) has stored thereon a lob value for assigning (S6) a result value to the biological sample (1), said lob value defining lower bound of the amount and/or the concentration of mutant copies of the target polynucleotide for classifying the biological sample (1) as including mutations of the target polynucleotide.
